(19) United States
(12) Patent Application Publication
(10) Pub. No.: US 2025/0179052 A1
(43) Pub. Date: Jun. 5, 2025

(54) PYRIMIDINYL-OXY-QUINOLINE BASED HERBICIDAL COMPOUNDS

(71) Applicant: SYNGENTA CROP PROTECTION AG, Basel (CH)

(72) Inventors: Vanitha ACHARYA, Corlim, Ilhas (IN); Sunil CHAKAVE, Corlim, Ilhas (IN); Mangala PHADTE, Corlim, Ilhas (IN)

(73) Assignee: SYNGENTA CROP PROTECTION AG, Basel (CH)

(21) Appl. No.: 18/842,685

(22) PCT Filed: Feb. 22, 2023
(86) PCT No.: PCT/EP2023/054447

§ 371 Date: Aug. 29, 2024

(30) Foreign Application Priority Data
Mar. 1, 2022 (IN) 202211010935

(51) Int. Cl. C07D 403/12; A01N 43/54; A01P 13/00; C07D 401/12

(52) U.S. Cl. CPC C07D 403/12; A01N 43/54; A01P 13/00; C07D 401/12

(57) ABSTRACT

The present invention relates to compounds of Formula (I), or an agronomically acceptable salt of said compounds wherein Y¹, Y², Z¹, Z², R¹, R², R⁵, R⁶, R⁹ and n are as defined herein. The invention further relates to herbicidal compositions which comprise a compound of Formula (I) and to the use of compounds of Formula (I) for controlling weeds, in particular in crops of useful plants.

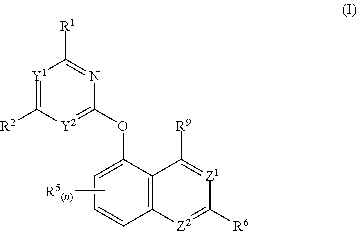

The present invention relates to novel herbicidal compounds, to processes for their preparation, to herbicidal compositions which comprise the novel compounds, and to their use for controlling weeds, in particular in crops of useful plants, or for inhibiting plant growth.

U.S. Pat. Nos. 4,952,235 and 5,068,394 disclose herbicidal (hetero)aryloxynaphthalene derivatives. WO2020/113554 discloses quinoline derivatives.

The present invention relates to heteroaryloxyquinazoline and heteroaryloxyquinoline compounds. Thus, according to the present invention there is provided a compound of Formula (I):

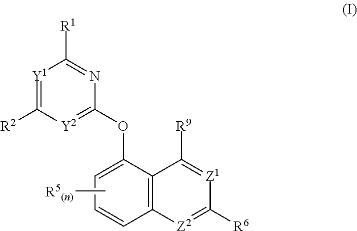

or an agronomically acceptable salt thereof,
wherein
Y¹ is N or CR³;
Y² is N or CR⁴;
with the proviso that Y¹ and Y² are not both N;
R¹ is selected from the group consisting of hydrogen, halogen, C₁-C₃alkyl and C₁-C₃haloalkyl;
R² is selected from the group consisting of hydrogen, halogen, hydroxyl, C₁-C₃alkyl, C₁-C₃alkoxy-, C₁-C₃haloalkoxy- and C₁-C₃haloalkyl;
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
each R⁵ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁶ is independently selected from the group consisting of hydrogen, halogen, —CN, hydroxy, C₁-C₆alkyl, C₁-C₆haloalkyl-, C₁-C₆alkoxy-, C₁-C₆haloalkoxy- and C₃-C₆cycloalkyl-;
Z¹ is N or CR⁷;
Z² is N or CR⁸;
R⁷ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁸ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁹ is hydrogen or methyl; and
n=0, 1 or 2.

C₁-C₄alkyl- and C₁-C₆alkyl-includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₆haloalkyl-includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl. C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₂alkoxy includes, for example, methoxy and ethoxy.

C₁-C₆haloalkoxy- and C₁-C₄haloalkoxy-include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₄alkenyl-includes, for example, —CH═CH₂ (vinyl) and —CH₂—CH═CH₂ (allyl).

C₂-C₄alkynyl-refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention there is provided a compound of Formula (I) wherein Y¹ is CR³ and Y² is N (in this embodiment R¹ and R² are preferably hydrogen); or Y¹ is CR³ and Y² is CR⁴ (in this embodiment R¹ and R² are preferably hydrogen); or Y¹ is N and Y² is CR⁴ (in this embodiment R¹ and R² are preferably hydrogen). In a more preferred embodiment of the present invention there is provided a compound of Formula (I) wherein Y¹ is CR³ and Y² is N wherein R³ is C₁-C₄alkyl (preferably methyl) or halo (preferably bromo or chloro), more preferably halo (preferably chloro).

In another embodiment of the present invention there is provided a compound of Formula (I) wherein Z¹ is CR⁷ (preferably CH) and Z² is CR⁸ (preferably CH); or Z¹ is CR⁷ (preferably CH) and Z² is N; or Z¹ is N and Z² is N; or Z¹ is N and Z² is CR⁸. In a more preferred embodiment Z¹ is N and Z² is N.

In another embodiment of the present invention there is provided a compound of Formula (I) wherein n=0. In another embodiment of the present invention there is provided a compound of Formula (I) wherein n=1, wherein R⁵ is selected from the group consisting of fluoro, chloro, bromo and CN.

In another embodiment of the present invention there is provided a compound of Formula (I) wherein R⁶ is selected from the group consisting of hydrogen, C₁-C₆alkyl (preferably methyl), C₁-C₆alkoxy-(preferably methoxy- or ethoxy-) and C₁-C₆haloalkyl-(preferably —CClF₂, —CHF₂ or CF₃). In another embodiment of the present invention there is provided a compound of Formula (I) wherein R⁶ is selected from the group consisting of hydrogen, C₁-C₆alkyl (preferably methyl) and C₁-C₆haloalkyl-(preferably —CHF₂ or CF₃). In another embodiment R⁶ is selected from the group consisting of hydrogen, methyl and CF₃.

In another embodiment of the present invention there is provided a compound of Formula (I) wherein R⁹ is hydrogen. In another embodiment there is provided a compound of Formula (I) wherein R⁹ is methyl.

In a particularly preferred embodiment of the present invention R¹ and R² are hydrogen, Y¹ is CR³ and Y² is N wherein R³ is C₁-C₄alkyl (preferably methyl) or halo (preferably chloro), n is 0, R⁶ is selected from the group consisting of hydrogen, C₁-C₆alkyl (preferably methyl), C₁-C₆alkoxy-(preferably methoxy or ethoxy) and C₁-C₆haloalkyl- (preferably —ClCF₂ or CF₃), Z¹ is N or CH, Z² is N, and R⁹ is hydrogen or methyl.

In a particularly preferred embodiment of the present invention R¹ and R² are hydrogen, Y¹ is CR³ and Y² is N wherein R³ is C₁-C₄alkyl (preferably methyl) or halo (preferably chloro), n is 0, R⁶ is selected from the group consisting of hydrogen, C₁-C₆alkyl (preferably methyl), C₁-C₆alkoxy-(preferably methoxy) and C₁-C₆haloalkyl-(preferably CF₃), Z¹ is N, Z² is N, and R⁹ is hydrogen or methyl.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester,4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H -indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, tetrahydrofuran-2-ylmethyl (2R)-2-[(4-amino-3,5-dichloro-6-fluoro-2-pyridyl)oxy]propanoate, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate and 6-amino-5-chloro-2-(4-chloro-2-fluoro-3-methoxy-phenyl)pyrimidine-4-carboxylic acid.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, —PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

Processes for preparation of compounds, e.g. a compound of formula (I) (which optionally can be an agrochemically acceptable salt thereof), are now described, and form further aspects of the present invention.

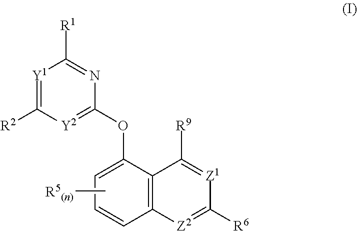

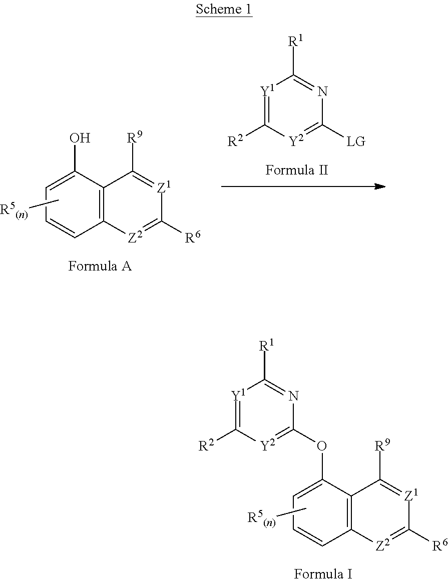

A compound of Formula I may be prepared from a compound of Formula A by reaction with a compound of Formula II (where LG represents a suitable leaving group such as F, Cl, Br or SO₂Me) in the presence of a suitable base and in a suitable solvent. Suitable bases may include NaH, K₂CO₃, Cs₂CO₃. Suitable solvents may include THF, CH₃CN or DMF. Compounds of Formula II are commercially available or may be prepared by known methods.

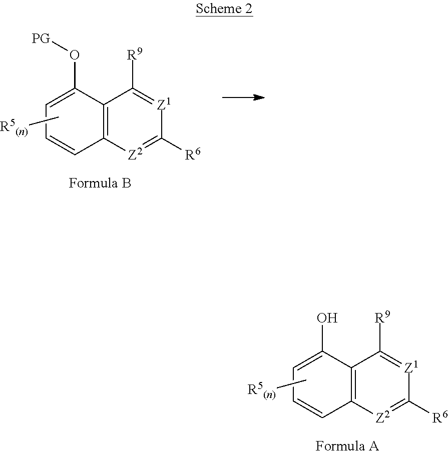

A compound of Formula A may be prepared from a compound of Formula B (where PG represents a suitable protecting group such as Me or Tf) by a deprotection reaction in a suitable solvent. Suitable deprotecting conditions may include BBr₃ or dodecanethiol/LiO^{t}Bu (for PG=Me) or K₂CO₃ (for PG=Tf). Suitable solvents may include DCM, DCE or CH₃CN.

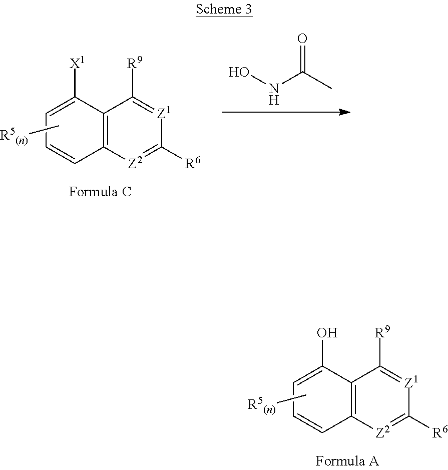

In an alternative method, a compound of Formula A may be prepared from compound of Formula C, (where X¹ is a leaving group such as Cl, Br, or F) by reaction with acetohydroxamic acid in the presence of a suitable base and in a suitable solvent (as described in Organic Letters (2016), 18 (9), 2244-2247). Suitable bases may include K₂CO₃. Suitable solvents may include DMSO.

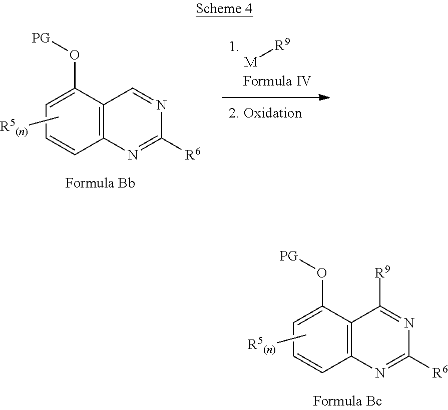

A compound of Formula Bc (a compound of Formula B where Z¹ and Z²=N) may be prepared from a compound of Formula Bb (a compound of Formula B where R⁹=H and Z¹ and Z²=N) in a two-step process. The first step involves reaction with a compound of Formula IV (where M is a suitable organometallic such as Li or MgHal), optionally in the presence of a suitable catalyst and in a suitable solvent. Suitable catalysts may include lanthanum (III) chloride bis(lithium chloride) complex. Suitable solvents may include THF. The second step involves reaction with a suitable oxidising agent in a suitable solvent. Suitable oxidising agents may include 3-dichloro-5,6-dicyano-1,4-benzoquinone or potassium ferricyanide. Suitable solvents may include THF or Et₂O/water. Compounds of Formula IV are commercially available or may be prepared by known methods.

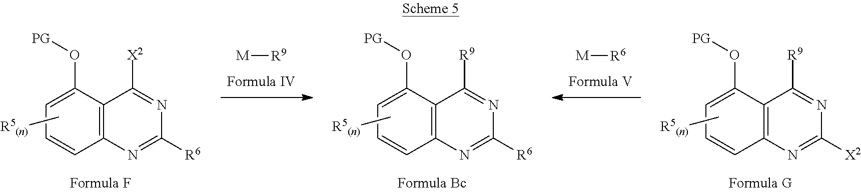

Alternatively, a compound of Formula Bc may be prepared from compound of Formula F or of Formula G (where X²=Cl or Br) by reaction with a compound of Formula IV or of Formula V (where M is a suitable organometallic such as Li or MgHal), optionally in the presence of a suitable catalyst and in a suitable solvent. Suitable catalysts may include copper chloride, iron (III) acetylacetonate and suitable solvents may include THF. Compounds of Formula V are commercially available or may be prepared by known methods.

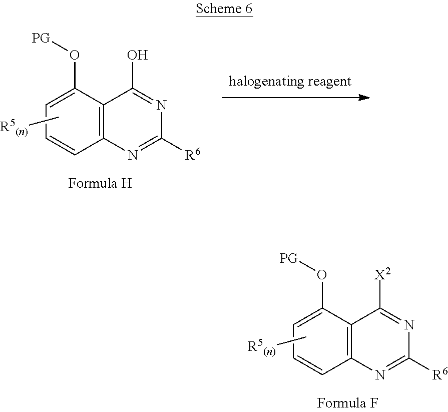

A Compound of Formula F may be prepared from compound of Formula H by reaction with a suitable halogenating agent. Suitable halogenating agents may include phosphorus oxychloride or phosphorus oxybromide.

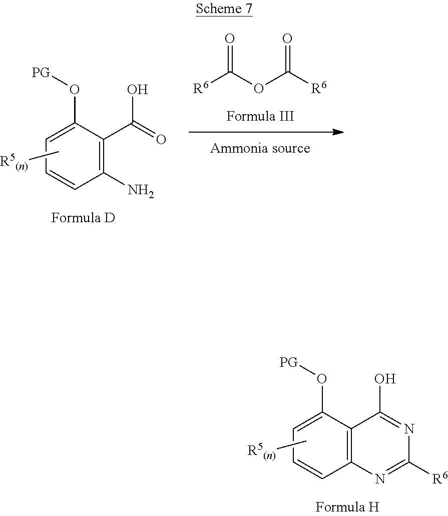

A compound of Formula H may be prepared from a compound of Formula D by reaction with a compound of Formula III and an ammonia source, in the presence of a suitable base and optionally in the presence of a suitable catalyst and in a suitable solvent. Suitable ammonia sources may include ammonium acetate. Suitable bases may include triethylamine. Suitable catalysts may include 4-dimethylaminopyridine. Suitable solvents may include toluene. Compounds of Formula D and of Formula III are commercially available or may be prepared by known methods.

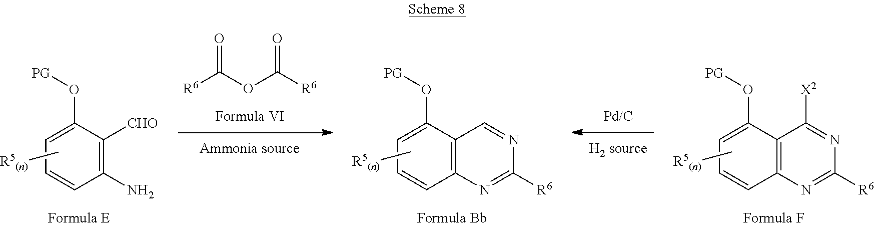

A compound of Formula Bb (a compound of Formula B where Z¹ and Z²=N) may be prepared from a compound of Formula E by reaction with a compound of Formula VI and an ammonia source, in the presence of a suitable base and optionally in the presence of a suitable catalyst and in a suitable solvent. Suitable ammonia sources may include ammonium acetate. Suitable bases may include triethylamine. Suitable catalysts may include 4-dimethylaminopyridine. Suitable solvents may include toluene. Compounds of Formula E and of Formula VI are commercially available or may be prepared by known methods.

Alternatively, a compound of Formula Bb may be prepared from compound of Formula F, by treatment with a suitable reducing agent in the presence of a suitable catalyst in a suitable solvent. Suitable reducing agents may include hydrogen gas. Suitable catalysts may include Pd/C. Suitable solvents may include methanol.

The following non-limiting examples provide specific synthesis methods for representative compounds of the present invention, as referred to in the Table below.

### EXAMPLE 1: SYNTHESIS OF 5-(5-chloropyrimidin-2-yl)oxy-4-methyl-2-(trifluoromethyl)quinazoline (COMPOUND 1.001)

### Step-1: Synthesis of 5-methoxy-4-methyl-2-(trifluoromethyl)quinazoline

To 0.6M lanthanum (III) trichloride bis(lithium chloride) complex in THF (30 ml) was added a mixture of 5-methoxy-2-(trifluoromethyl) quinazoline (3 g, 13.15 mmol) in tetrahydrofuran (60 mL) under a nitrogen atmosphere. The mixture was cooled to 0° C. and 3.0M THF solution of methylmagnesiumbromide (13.15 ml, 39.44 mmol) was added. The mixture was stirred at room temperature for 1 h. The reaction mixture was cooled to 0° C. and then slowly quenched with saturated ammonium chloride solution with stirring and extracted with ethyl acetate. The organics were combined, dried and concentrated. The residue was dissolved in tetrahydrofuran (40 mL) and 2,3-dichloro-5,6-dicyano-1,4-benzoquinone (7.59 g, 32.76 mmol) was added in one portion at 0° C. The reaction mixture was stirred at room temperature for 2 h then diluted with aqueous saturated sodium bicarbonate solution and extracted with ethyl acetate. The organics were combined, washed with brine, dried and concentrated. The residue was purified by flash column chromatography to afford 5-methoxy-4-methyl-2-(trifluoromethyl) quinazoline (2.5 g, 63%) as an off white solid.

¹H NMR (400 MHZ, CHLOROFORM-d) δ ppm 7.85 (t, 1H), 7.71 (d, 1H), 7.06 (d, 1H), 4.04 (s, 3H), 3.16 (s, 3H)

### Step-2: Synthesis of 4-methyl-2-(trifluoromethyl)quinazolin-5-ol

A solution of 1-dodecanethiol (0.85 g, 4.12 mmol) and lithium tert-butoxide (0.34 g, 4.13 mmol) in N,N-dimethylformamide (5.89 mL) was stirred at room temperature for 15 minutes. 5-methoxy-4-methyl-2-(trifluoromethyl) quinazoline (0.5 g, 2.06 mmol) was added and the reaction mixture was heated at 100° C. for 4 hours. Upon cooling to room temperature, the reaction was quenched with water, acidified with 2M HCl and extracted with ethyl acetate. The organics were combined, washed with brine, dried and concentrated. The residue was purified by flash column chromatography to afford 4-methyl-2-(trifluoromethyl) quinazolin-5-ol (0.47 g, 99%) as a light yellow solid.

¹H NMR (400 MHZ, METHANOL-d4) δ ppm 7.81 (t, 1H), 7.53 (dd, 1H), 7.11 (dd, 1H), 3.16 (s, 3H)

### Step-3: Synthesis of 5-(5-chloropyrimidin-2-yl)oxy-4-methyl-2-(trifluoromethyl) quinazoline (Compound 1.001)

To a solution of 4-methyl-2-(trifluoromethyl) quinazolin-5-ol (0.75 g, 3.29 mmol) in isopropanol (15 ml) was added potassium carbonate (0.91 g, 6.57 mmol) and 5-chloro-2-methylsulfonyl-pyrimidine (1.27 g, 6.57 mmol). The reaction mixture was heated to 50° C. for 16 h then cooled to room temperature, diluted with water and extracted with ethyl acetate. The organics were combined, washed with brine, dried and concentrated. The residue was purified by flash column chromatography to afford 5-(5-chloropyrimidin-2-yl)oxy-4-methyl-2-(trifluoromethyl) quinazoline (1.0 g, 89%) as an off white solid.

¹H NMR (400 MHZ, CHLOROFORM-d) δ ppm 8.52 (s, 2H), 8.13 (d, 1H), 8.00 (t, 1H), 7.49 (d, 1H), 3.01 (s, 3H)

### Example 2: Synthesis of 5-(5-chloropyrimidin-2-yl)oxy-2-(trifluoromethyl) quinazoline (Compound 1.002)

### Step 1: Synthesis of 5-methoxy-2-(trifluoromethyl)quinazoline

A stirred solution of 2-amino-6-methoxy-benzaldehyde (1.00 g, 6.62 mmol) and 4-dimethylaminopyridine (0.082 g, 0.66 mmol) in toluene (20 mL) was cooled to 0° C. was triethylamine (2.04 mL, 14.6 mmol) was added. trifluoroacetic acid anhydride (1.43 mL, 9.92 mmol) was added drop wise and the reaction mixture was allowed to warm to room temperature and stirred for 18 hours. Ammonium acetate (1.16 g, 14.6 mmol) was added and the reaction was heated at 90° C. for 24 hours. Upon cooling, the reaction was diluted with water and extracted with ethyl acetate. The combined organics were dried and concentrated. The residue was purified by flash column chromatography to afford 5-methoxy-2-(trifluoromethyl) quinazoline (0.69 g, 46%) as a pale yellow solid. ¹H NMR (400 MHZ, CDCl₃) δ 9.87 (s, 1H), 7.95 (t, 1H), 7.75 (d, 1H), 7.08 (d, 1H), 4.09 (s, 3H)

### Step-2: Synthesis of 2-(trifluoromethyl)quinazolin-5-ol

To a solution of 1-dodecanethiol (2.4 g, 11 mmol) in N,N-dimethylformamide (16 mL) was added lithium tert-butoxide (0.93 g, 11 mmol) followed by 5-methoxy-2-(trifluoromethyl)-4-[2-(trifluoromethyl)pyrimidin-5-yl]quinazoline (1.3 g, 5.7 mmol). The reaction mixture was heated to 100° C. for 2.3 h then cooled to room temperature, quenched with water, acidified with 2N HCl and extracted with ethyl acetate. The combined organics were dried and concentrated. The residue was purified by flash column chromatography to afford 2-(trifluoromethyl) quinazolin-5-ol (0.64 g, 52%) as a white solid.

¹H NMR (400 MHZ, MeOD) δ=9.85 (s, 1H), 7.94 (t, 1H), 7.60 (d, 1H), 7.15 (d, 1H)

### Step-3: Synthesis of 5-(5-chloropyrimidin-2-yl)oxy-2-(trifluoromethyl)quinazoline (1.002)

To a solution of 2-(trifluoromethyl) quinazolin-5-ol (0.43 g, 2.01 mmol) in isopropyl alcohol (6.45 mL) was added potassium carbonate (0.83 g, 6.02 mmol) and 5-chloro-2-methylsulfonyl-pyrimidine (0.58 g, 3.01 mmol). The reaction mixture was heated at 50° C. for 16 h then cooled to room temperature, quenched with water, extracted with ethyl acetate, dried and concentrated. The residue was purified by flash column chromatography to afford 5-(5-chloropyrimidin-2-yl)oxy-2-(trifluoromethyl) quinazoline (0.34 g, 52%) as a white solid.

¹H NMR (400 MHZ, CHLOROFORM-d) δ ppm 9.68 (s, 1H) 8.54 (s, 2H) 8.15 (d, 1H) 8.10 (t, 1H) 7.63 (dd, 1H)

### Example 3: Preparation of 5-(5-bromopyrimidin-2-yl)oxy-2-[chloro(difluoro)methyl]quinazoline (1.055)

### Step-1: Preparation of 2-[chloro(difluoro)methyl]-5-methoxy-quinazoline (I1)

To a solution of 2-amino-6-methoxybenzaldehyde (1.93 g, 12.8 mmol) in toluene (38.3 mL) was added dimethyl amino pyridine (0.158 g, 1.28 mmol) and ammonium acetate (4.46 g, 56.2 mmol). The reaction mixture was cooled to 0° C. and chlorodifluoroacetic anhydride (6.33 g, 4.54 mL) was added dropwise followed by triethylamine (10.8 mL, 76.6 mmol) over a period of 15 min. This was allowed to warm to room temp and then stirred at 70° C. for 16 h. The reaction mass was quenched with ice cold water (500 mL) and stirred vigorously for 20 min, extracted with ethyl acetate (3×100 mL). Organic layer was washed with saturated bicarbonate solution (50 mL) and then with brine solution (50 mL), dried over anhydrous sodium sulphate, filtered, and concentrated to get the crude material. The crude was purified by silica (200-400 mesh) using ethyl acetate and cyclohexane (20:80) in combiFlash to afford 2-[chloro(difluoro)methyl]-5-methoxy-quinazoline I1 (1.68 g, 54%) as a light yellow solid. ¹H NMR (400 MHZ, CDCl₃) δ ppm 9.85 (s, 1H), 7.93 (t, 1H), 7.72 (d, 1H), 7.05 (d, 1H), 4.08 (s, 3H).

### Step-2: Preparation of 2-[chloro(difluoro)methyl]quinazolin-5-ol (I2)

To a 30 mL microwave vial were added 2-[chloro(difluoro)methyl]-5-methoxy-quinazoline 11 (1.0 g, 4.1 mmol) and pyridinium hydrochloride (9.7 g, 82 mmol). The reaction mass was irradiated with microwave radiation for 1 h at 180° C. The reaction mixture diluted with water (100 mL) and extracted with ethyl acetate (3×100 mL). Organic layer was washed with saturated bicarbonate solution (50 ml) and then with brine (50 mL), dried over anhydrous sodium sulphate, filtered, and concentrated to get the crude material. The residue was purified by silica (200-400 mesh) using ethyl acetate and cyclohexane (30:70) in combiFlash to afford 2-[chloro(difluoro)methyl]quinazolin-5-ol I2 (0.410 g, 42%) as a beige solid. ¹H NMR (400 MHZ, DMSO-d6) δ ppm 11.50 (s, 1H), 9.84 (s, 1H), 7.98 (t, 1H), 7.58 (d, 1H), 7.20 (dd, 1H).

### Step-3: Preparation of 5-(5-bromopyrimidin-2-yl)oxy-2-[chloro(difluoro)methyl]quinazoline (1.055)

To a solution of 2-[chloro(difluoro)methyl]quinazolin-5-ol 12 (0.25 g, 1.08 mmol) in acetonitrile (5 mL) were added potassium carbonate (0.45 g, 3.25 mmol) and 5-bromo-2-chloro-pyrimidine (0.42 g, 2.17 mmol). The reaction mass was irradiated with microwave radiation for 1 h at 120° C. The reaction mixture diluted with water (50 mL) and extracted with ethyl acetate (3×100 mL). The organic layer was washed with saturated bicarbonate solution (50 mL) and then with brine solution (50 mL), dried over anhydrous sodium sulphate, filtered, and concentrated to get the crude material. The residue was purified by silica (200-400) using the ethyl acetate and cyclohexane (10:90) in combiFlash to afford 5-(5-bromopyrimidin-2-yl)oxy-2-[chloro(difluoro)methyl]quinazoline 1.055 (0.300 g, 67%) as off-white solid. ¹H NMR (400 MHZ, CDCl₃) δ ppm 9.69 (s, 1H), 8.63 (s, 2H), 8.08-8.17 (m, 2H), 7.62 (dd, 1 H).

### Example 4: Preparation of 2-[chloro(difluoro)methyl]-5-(5-methylpyrimidin-2-yl)oxy-quinazoline (Compound 1.056)

To a solution of 5-(5-bromopyrimidin-2-yl)oxy-2-[chloro(difluoro)methyl]quinazoline 1.055 (0.15 g, 0.39 mmol) in toluene (1.8 mL) and water (0.6 mL) were added potassium carbonate (0.16 g, 1.16 mmol) and methylboronic acid (0.039 g, 0.58 mmol). The mixture was degassed by bubbling nitrogen for 15 min. To this mixture, 1,1′-bis(diphenylphosphino)ferrocene-palladium (II) dichloride dichloromethane complex (0.016 g, 0.019 mmol) was added and it was heated to 90° C. for 15 h. The reaction mass diluted with water (100 mL) and extracted with ethyl acetate (3×100 mL). Organic layer was washed with saturated bicarbonate solution (50 mL) and then with brine solution (50 mL), dried over anhydrous sodium sulphate, filtered, and concentrated to get crude material. The residue was purified by (200-400) silica using ethyl acetate and cyclohexane (20:80) to afford 2-[chloro(difluoro)methyl]-5-(5-methylpyrimidin-2-yl)oxy-quinazoline 1.056 (0.038 g, 29%) as off-white solid. ¹H NMR (400 MHZ, CDCl₃) δ ppm 9.70 (s, 1H), 8.41 (s, 2H), 8.05-8.13 (m, 2H), 7.59 (dd, 1 H), 2.32 (3 H, s).

### Example 5: Preparation of 2-[chloro(difluoro)methyl]-5-(5-fluoropyrimidin-2-yl)oxy-quinazoline (Compound 1.061)

To a solution of 2-[chloro(difluoro)methyl]quinazolin-5-ol I2 (0.1 g, 0.434 mmol) in acetonitrile (2 mL) was added potassium carbonate (0.18 g, 1.30 mmol), followed by 2-chloro-5-fluoropyrimidine (0.115 g, 0.87 mmol). It was heated at 120° C. in microwave for 1 h. The reaction mass was cooled to room temperature, diluted with water (25 mL) and ethyl acetate (50 mL). The layers were separated. The aqueous layer was extracted with ethyl acetate (2×50 mL), the combined organic layer was washed with brine solution (25 mL) and concentrated to get crude material. The residue was purified by silica (200-400) using ethyl acetate and cyclohexane (15:85) in combi flash to afford 2-[chloro(difluoro)methyl]-5-(5-fluoropyrimidin-2-yl)oxy-quinazoline 1.061 (0.11 g, 74%) as beige solid. ¹H NMR (400 MHZ, CDCl₃) δ ppm 9.69 (s, 1H), 8.45 (s, 2H), 8.05-8.14 (m, 2H), 7.59 (dd, 1H).

### Example 6: Preparation of 5-(5-chloropyrimidin-2-yl)oxy-2-ethoxy-quinazoline (Compound 1.063)

### Step-1: Preparation of (2-amino-6-methoxy-phenyl) methanol (I4)

To a solution of 2-amino-6-methoxybenzoic acid (10 g, 56.83 mmol) in tetrahydrofuran (50 mL) at 0° C. was added 2 M solution of lithium aluminium hydride (56.8 mL, 113.7 mmol) drop wise over 15 minutes. The reaction mixture was slowly warmed to room temperature and stirred until completion. This was cooled to 0° C. and then quenched with 10 ml of water and 20 ml of 1 M sodium hydroxide. The emulsion was extracted with ethyl acetate (100 mL×3). The organic layer was washed with brine (100 mL) and dried over anhydrous sodium sulphate, filtered, and concentrated to get the crude product. This was purified by column chromatography on silica gel using ethyl acetate and cyclohexane (40:60) to afford (2-amino-6-methoxy-phenyl) methanol 14 (6.8 g, 78%) as a pale-yellow oil. ¹H NMR (400 MHZ, CDCl₃) δ ppm 7.06 (t, 1H), 6.35 (d, 1H), 6.32 (d, 1H), 4.78 (s, 2H), 3.79 (s, 3H).

### Step-2: Preparation of 2-amino-6-methoxy-benzaldehyde (I5)

To a solution of (2-amino-6-methoxy-phenyl) methanol I4 (2.2 g, 13 mmol) in 1,2-dichloroethane (110 mL) was added MnO₂ (13 g, 130 mmol) powder. The reaction mixture was heated to 40° C. for 24 h. The mixture was filtered through the celite bed and the solvent was removed under reduced pressure to afford the crude product which was purified by column chromatography on silica gel using ethyl acetate and cyclohexane (40:60) to afford 2-amino-6-methoxy-benzaldehyde I5 (1.2 g, 61%) as a brown solid. ¹H NMR (400 MHZ, CDCl₃) δ ppm 10.42 (s, 1H), 7.20 (t, 1H), 6.20 (d, 1 H), 6.11 (d, 1H), 3.85 (s, 3H).

### Step-3: Preparation of 2,2,2-trichloro-N-(2-formyl-3-methoxy-phenyl)acetamide (I6)

To a solution of 2-amino-6-methoxy-benzaldehyde I5 (1.5 g, 9.9 mmol) in acetonitrile (30 mL) were added N,N-dimethylpyridin-4-amine (1.8 g, 15 mmol) and 2,2,2-trichloroacetyl chloride (1.2 mL, 11 mmol) dropwise at 0° C. The reaction mixture was then warmed to room temperature and stirred overnight. Reaction mixture was cooled to 0° C., quenched by adding water (50 mL) and then extracted with ethyl acetate (100 ml×3). Organic layer was washed with brine (100 mL) and dried over anhydrous sodium sulphate, filtered, and concentrated under reduced pressure to get the crude product 2,2,2-trichloro-N-(2-formyl-3-methoxy-phenyl) acetamide 16 as a brown solid (2.5 g, 85%) which was used directly for the next step.

¹H NMR (400 MHZ, CDCl₃) δ ppm 10.55 (s, 1H), 8.24 (d, 1H), 7.61 (t, 1H), 6.80 (d, 1H), 3.96 (s, 3H).

### Step-4: Preparation of 5-methoxyquinazolin-2-ol (I7)

To a solution of 2,2,2-trichloro-N-(2-formyl-3-methoxy-phenyl) acetamide I6 (2.5 g, 8.4 mmol) in toluene (50 mL) was added ammonium acetate (3.2 g, 42 mmol) and the reaction mass was heated to reflux for 18 h. Reaction mixture was quenched in water (100 mL). The precipitate obtained was filtered and dried over high vacuum to obtain 5-methoxyquinazolin-2-ol 17 (0.800 g, 54%) as a brown solid. ¹H NMR (400 MHz, DMSO-d6) δ ppm 11.83 (br s, 1H), 9.33 (s, 1H), 7.64 (t, 1H), 6.80 (dd, 2H), 3.94 (s, 3H).

### Step-5: Preparation of 2-chloro-5-methoxy-quinazoline (I8)

To a solution of 5-methoxyquinazolin-2-ol I7 (1 g, 5.67 mmol) and phosphoryl trichloride (13.50 mL, 141.91 mmol) was added N,N-diisopropylethylamine (0.69 mL, 3.97 mmol) dropwise at 0° C. The reaction mixture was then warmed to room temperature and heated to reflux for 4 h. Reaction mixture was cooled to room temperature and concentrated to obtain a yellow solid which was slowly dissolved into ice-cold saturated solution of sodium bicarbonate. After stirring for 15 min, this was extracted with ethyl acetate (3×100 mL) and the organic layer was washed with brine (100 mL), dried over anhydrous sodium sulphate, filtered, and concentrated to get the crude product. The crude product was purified by column chromatography on silica gel using ethyl acetate and cyclohexane (40:60) to afford 2-chloro-5-methoxy-quinazoline 18 (0.6 g, 54%) as a white solid. ¹H NMR (400 MHZ, CDCl₃) δ ppm 9.61 (s, 1H), 7.85 (t, 1H), 7.53 (d, 1H), 6.94 (d, 1H), 4.05 (s, 3H).

### Step-6: Preparation of 2-bromoquinazolin-5-ol (I9)

To a solution of 2-chloro-5-methoxy-quinazoline 18 (0.4 g, 2.05 mmol) in 1,2-dichloroethane (15 mL) was added 1 M solution of BBr₃ (8.2 mL, 8.2 mmol) dropwise at 0° C. The reaction mixture was warmed to room temperature and heated to 60° C. for 12 h. Reaction mixture was cooled to room temperature and quenched with water (50 mL) and extracted with ethyl acetate (3×50 mL). Organic layer was washed with brine (50 mL) and dried over anhydrous sodium sulphate filtered and concentrated under reduced pressure to get the crude product which was purified by column chromatography on silica gel using ethyl acetate and cyclohexane (80:20) to afford 2-bromoquinazolin-5-ol I9 (0.250 g, 54%) as a yellow solid. ¹H NMR (400 MHZ, CDCl₃) δ ppm 10.40 (br s, 1H), 9.45 (d, 1H), 7.61-7.78 (m, 1H), 7.31 (dd, 1H), 6.94 (dd, 1 H).

### Step-7: preparation of 2-ethoxyquinazolin-5-ol (I10)

To a solution of 2-bromoquinazolin-5-ol I9 (0.20 g, 0.89 mmol) in ethanol (2 mL) were added 21% solution of sodium ethoxide in ethanol (1.44 g, 4.44 mmol) followed by potassium carbonate (0.20 g, 1.42 mmol). This was then heated to 50° C. for 12 h. Reaction mixture was quenched with NH₄Cl (20 mL) and extracted with ethyl acetate (3×20 mL). Organic layer was washed with brine (20 mL) and dried over anhydrous sodium sulphate filtered and concentrated under reduced pressure to get the crude product which was purified by column chromatography on silica gel using ethyl acetate and cyclohexane (90:10) to afford 2-ethoxyquinazolin-5-ol I10 as a yellow solid (0.090 g, 53%). ¹H NMR (400 MHZ, CDCl₃) δ ppm 9.62 (s, 1H), 7.61 (t, 1H), 7.31 (d 1 H), 6.83 (d, 1H), 4.57 (q, 2H), 1.48 (t, 3H).

### Step-8: Preparation of 5-(5-chloropyrimidin-2-yl)oxy-2-ethoxy-quinazoline (Compound 1.063)

To a solution of 2-ethoxyquinazolin-5-ol I10 (0.09 g, 0.47 mmol) in isopropyl alcohol (4.5 mL) was added 5-chloro-2-methylsulfonyl-pyrimidine (0.14 g, 0.71 mmol) followed by potassium carbonate (0.20 g, 1.42 mmol) and then heated to 55° C. for 18 h. Reaction mixture was quenched with water (20 mL) then extracted with ethyl acetate (20 mL×3). Organic layer was washed with brine (20 mL) and dried over anhydrous sodium sulfate filtered and concentrated under reduced pressure to get the crude product which was purified by column chromatography on silica gel using ethyl acetate and cyclohexane (30:70) to afford 5-(5-chloropyrimidin-2-yl)oxy-2-ethoxy-quinazoline 1.063 (0.05 g, 35%) as a white solid. ¹H NMR (400 MHZ, CDCl₃) δ ppm 9.34 (s, 1H), 8.52 (s, 2H), 7.84 (dd, 1H), 7.74 (d, 1H), 7.22 (d, 1H), 4.58 (q, 2H), 1.45-1.55 (m, 3H).

### Example 7: Preparation of 5-(5-chloropyrimidin-2-yl)oxy-2 (trifluoromethyl) quinoline (1.020)

### Step-1: Preparation of 8-bromo-5-methoxy-2-(trifluoromethyl)quinolin-4-ol (I11)

A mixture of ethyl 4,4,4-trifluoro-3-oxo-butanoate (17.31 g, 94.03 mmol) and polyphosphoric acid (80 g) was heated to 100° C. for 30 min. To this, 2-bromo-5-methoxyaniline (20 g, 94.03 mmol) was added and heated again to 150° C. for 5 h. Reaction mixture was cooled to room temperature then a solution of sodium hydroxide (20 g) in water (100 mL) was added to the reaction mixture dropwise at 0° C. The obtained precipitate was filtered, and the filtrate was acidified by using concentrated HCl. The precipitate was washed with water (50 mL). This was then re-dissolved in acetonitrile and concentrated to give 8-bromo-5-methoxy-2-(trifluoromethyl)quinolin-4-ol 111 (26 g, 85%) as brown solid. ¹H NMR (400 MHZ, DMSO-d6) δ ppm 11.35 (s, 1H), 8.11 (d, 1H), 7.27 (s, 1H), 7.05 (d, 1H), 3.99 (s, 3 H).

### Step-2: Preparation of 5-methoxy-2-(trifluoromethyl)quinolin-4-ol (I12)

A solution of 8-bromo-5-methoxy-2-(trifluoromethyl)quinolin-4-ol I11 (8.8 g, 27 mmol) in tetrahydrofuran (88 mL) was cooled to −78° C. and n-butyl lithium 2.5 M in hexane (33 mL, 82 mmol) was added drop wise. Reaction mixture was then stirred for 2 h at the same temperature and methanol (44 mL) was added. This was slowly warmed to room temperature. The reaction mixture was quenched with NH₄Cl (100 mL) and then extracted with ethyl acetate (3×100 mL). Organic layer was washed with brine (100 mL) and dried over anhydrous sodium sulphate filtered and concentrated under reduced pressure to get the crude product which was purified by column chromatography on silica gel using ethyl acetate and cyclohexane (30:70) to afford 5-methoxy-2-(trifluoromethyl)quinolin-4-ol I12 (3.5 g, 52%) as a yellow solid. ¹H NMR (400 MHZ, DMSO-d6) δ ppm 10.99 (br s, 1H), 7.66-7.79 (m, 1H), 7.51-7.66 (m, 1 H), 7.02-7.24 (m, 2H), 4.00 (s, 3H).

### Step-3: Preparation of 4-bromo-5-methoxy-2-(trifluoromethyl)quinoline (I13)

To a solution of 5-methoxy-2-(trifluoromethyl)quinolin-4-ol I12 (3.3 g, 14 mmol) in toluene (35 mL) was added phosphoryl bromide (6.1 g, 20 mmol) and heated to 100° C. for 30 min then gradually heating was increased to 140° C. and stirred at this temperature for 5 h. The reaction mixture was cooled to 0° C. and quenched by adding sodium bicarbonate solution till pH 7 (checked by pH paper). The mixture was extracted with ethyl acetate (3×100 mL). Organic layer was washed with brine (100 mL) and dried over anhydrous sodium sulfate filtered and concentrated under reduced pressure to get the crude product which was purified by column chromatography on silica gel using ethyl acetate and cyclohexane (30:70) to afford 4-bromo-5-methoxy-2-(trifluoromethyl)quinoline I13 (3 g, 72%) as a white solid. 1H NMR (400 MHZ, CDCl₃) δ ppm 7.97 (s, 1H), 7.82 (d, 1H), 7.72 (t, 1H), 7.05 (d, 1H), 4.00 (s, 3H).

### Step-4: Preparation of 5-methoxy-2-(trifluoromethyl)quinoline (I14)

To a solution of 4-bromo-5-methoxy-2-(trifluoromethyl)quinoline I13 (3 g, 9.8014 mmol) in methanol (60 mL) was added 5% palladium on carbon (wet) (0.6 g) and stirred at room temperature under hydrogen pressure (using hydrogen balloon). Reaction mixture was then filtered through celite bed and washed with ethyl acetate (50 mL). Removal of solvent under reduced pressure afforded crude product which was purified by column chromatography on silica gel using ethyl acetate and cyclohexane (20:80) to afford 5-methoxy-2-(trifluoromethyl)quinoline I14 (2.1 g, 94%) as a white solid. ¹H NMR (400 MHZ, CDCl₃) δ ppm 8.78 (d, 1H), 7.79-7.83 (m, 1 H), 7.70-7.75 (m, 2H), 6.97 (d, 1H), 4.05 (s, 3H).

### Step-5: Preparation of 2-(trifluoromethyl)quinolin-5-ol (I15)

To a solution of 5-methoxy-2-(trifluoromethyl)quinoline I14 (2.0 g, 8.8 mmol) in dichloromethane (50 mL) was added 1 M solution of BBr₃ (11 mL, 26 mmol) dropwise at 0° C. The reaction mixture was then warmed to room temperature and stirred for 12 h and then heated to 40° C. for 1 h. The reaction mixture was cooled to 0° C. and quenched by adding a solution of sodium bicarbonate till pH 7 (checked with pH paper). The solution was extracted with ethyl acetate (3×50 mL). Organic layer was washed with brine (50 mL) and dried over anhydrous sodium sulphate filtered and concentrated under reduced pressure to get the crude product which was purified by column chromatography on silica gel using ethyl acetate and cyclohexane (30:70) to afford 2-(trifluoromethyl)quinolin-5-ol I15 (1.2 g, 64%) as a yellow solid. ¹H NMR (400 MHZ, DMSO-d6) δ ppm 10.91 (s, 1H), 8.80 (d, 1H), 7.88 (d, 1H), 7.73 (dd, 1 H), 7.61 (d, 1H), 7.10 (dd, 1H)

### Step-6: Preparation of 5-(5-chloropyrimidin-2-yl)oxy-2 (trifluoromethyl)quinoline (1.020)

To a solution of 2-(trifluoromethyl)quinolin-5-ol I15 (0.30 g, 1.41 mmol) in acetonitrile (6 mL) was added 2,5-dichloropyrimidine (0.42 g, 2.81 mmol) followed by potassium carbonate (0.58 g, 4.22 mmol) and then heated to 120° C. for 1 h. Reaction mixture was quenched with water (20 mL) then extracted with ethyl acetate (20 mL×3). Organic layer was washed with brine (50 mL) and dried over anhydrous sodium sulphate filtered and concentrated under reduced pressure to get the crude product which was purified by column chromatography on silica gel using ethyl acetate and cyclohexane (30:70) to afford 5-(5-chloropyrimidin-2-yl)oxy-2 (trifluoromethyl) quinoline 1.020 (0.360 g, 74%) as a yellow solid. ¹H NMR (400 MHZ, CDCl₃) δ ppm 8.51 (s, 2H), 8.49 (d, 1H), 8.20 (d, 1H), 7.88 (t, 1H), 7.74 (d, 1H), 7.52 (d, 1H).

TABLE 1
Examples of herbicidal compounds of the present invention.

| | | NMR (400 MHz, CDCl₃ unless otherwise |
|---|---|---|
| COMPOUND | STRUCTURE | stated) |
| 1.001 | 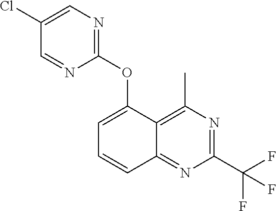 [SMILES: Cc1nc(C(F)(F)F)nc2cccc(Oc3ncc(Cl)cn3)c12] | 8.52 (s, 2 H), 8.13 (d, 1 H), 8.00 (t, 1 H), 7.49 (d, 1 H), 3.01 (s, 3 H) |
| 1.002 | 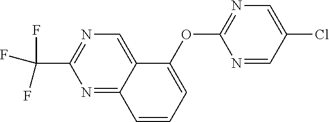 [SMILES: FC(F)(F)c1ncc2c(Oc3ncc(Cl)cn3)cccc2n1] | 9.68 (s, 1 H) 8.54 (s, 2 H) 8.07-8.17 (m, 2 H) 7.63 (dd, 1 H) |
| 1.003 | 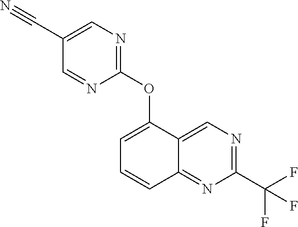 [SMILES: N#Cc1cnc(Oc2cccc3nc(C(F)(F)F)ncc23)nc1] | 9.63 (s, 1 H) 8.87 (s, 2 H) 8.21 (d, 1 H) 8.13 (dd, 1 H) 7.65 (dd, 1 H) |
| 1.004 | 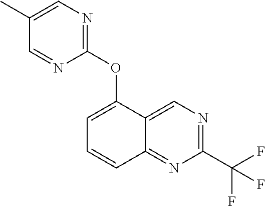 [SMILES: Cc1cnc(Oc2cccc3nc(C(F)(F)F)ncc23)nc1] | 9.70 (s, 1 H) 8.42 (s, 2 H) 8.11 (s, 1 H) 8.09 (d, 1 H) 7.61 (dd, 1 H) 2.32 (s, 3 H) |
| 1.005 | 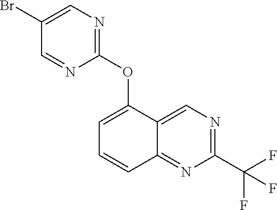 [SMILES: FC(F)(F)c1ncc2c(Oc3ncc(Br)cn3)cccc2n1] | 9.68 (s, 1 H) 8.62 (s, 2 H) 8.16 (d, 1 H) 8.10 (dd, 1 H) 7.63 (dd, 1 H) |
| 1.006 | 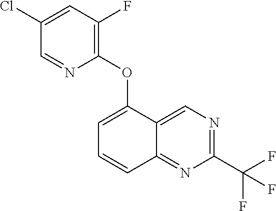 [SMILES: Fc1cc(Cl)cnc1Oc1cccc2nc(C(F)(F)F)ncc12] | 9.78 (s, 1 H) 8.03-8.11 (m, 2 H) 7.91 (d, 1 H) 7.67 (dd, 1 H) 7.57 (dd, 1 H) |
| 1.007 | 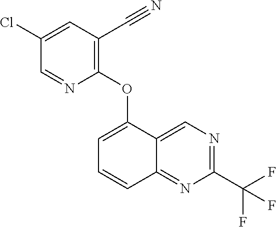 [SMILES: N#Cc1cc(Cl)cnc1Oc1cccc2nc(C(F)(F)F)ncc12] | 9.93 (s, 1 H) 8.82 (d, 1 H) 8.41 (d, 1 H) 8.29 (dd, 1 H) 8.20 (d, 1H) 7.92 (dd, 1 H) |
| 1.008 | 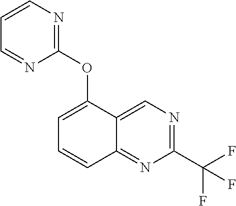 [SMILES: FC(F)(F)c1ncc2c(Oc3ncccn3)cccc2n1] | 9.70 (s, 1 H) 8.61 (d, 2 H) 8.08-8.16 (m, 2 H) 7.64 (d, 1 H) 7.18 (t, 1 H) |
| 1.009 | 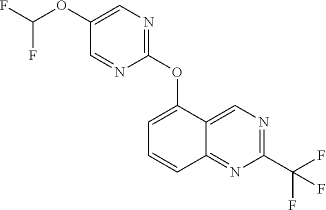 [SMILES: FC(F)Oc1cnc(Oc2cccc3nc(C(F)(F)F)ncc23)nc1] | 9.70 (s, 1 H), 8.47 (s, 2 H), 8.07-8.16 (m, 2 H), 7.63 (d, 1 H), 6.58 (t, 1 H) |
| 1.010 | 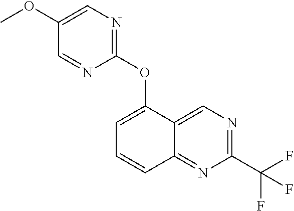 [SMILES: COc1cnc(Oc2cccc3nc(C(F)(F)F)ncc23)nc1] | |
| 1.011 | 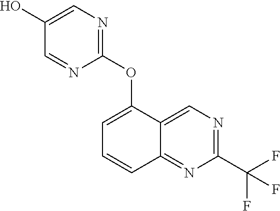 [SMILES: Oc1cnc(Oc2cccc3nc(C(F)(F)F)ncc23)nc1] | |
| 1.012 | 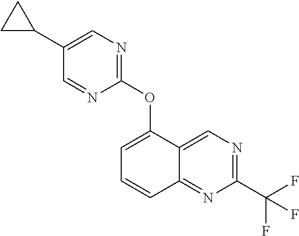 [SMILES: FC(F)(F)c1ncc2c(Oc3ncc(C4CC4)cn3)cccc2n1] | 9.69 (s, 1 H), 8.32 (s, 2 H), 8.04-8.09 (m, 2 H), 7.59 (d, 1 H), 1.87 (m, 1 H), 1.09 (m, 2 H), 0.75 (m, 2 H) |
| 1.014 | 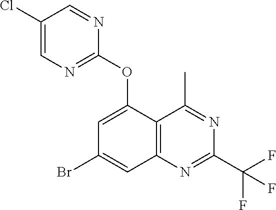 [SMILES: Cc1nc(C(F)(F)F)nc2cc(Br)cc(Oc3ncc(Cl)cn3)c12] | |
| 1.015 | 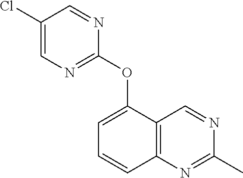 [SMILES: Cc1ncc2c(Oc3ncc(Cl)cn3)cccc2n1] | |
| 1.016 | 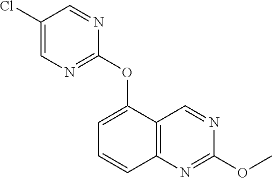 [SMILES: COc1ncc2c(Oc3ncc(Cl)cn3)cccc2n1] | 9.34 (d, 1 H), 8.51 (s, 2 H), 7.84 (t, 1 H), 7.75 (d, 1 H), 7.23 (d, 1 H), 4.14 (s, 3 H) |
| 1.017 | 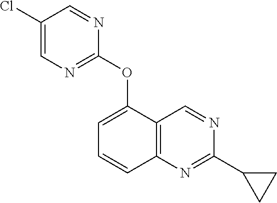 [SMILES: Clc1cnc(Oc2cccc3nc(C4CC4)ncc23)nc1] | |
| 1.018 | 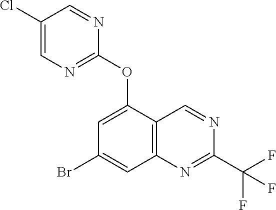 [SMILES: FC(F)(F)c1ncc2c(Oc3ncc(Cl)cn3)cc(Br)cc2n1] | |
| 1.019 | 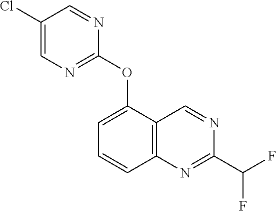 [SMILES: FC(F)c1ncc2c(Oc3ncc(Cl)cn3)cccc2n1] | 9.65 (s, 1 H), 8.52 (s, 2 H), 8.02-8.11 (m, 2 H), 7.55-7.58 (m, 1 H), 6.83 (t, 1 H) |
| 1.020 | 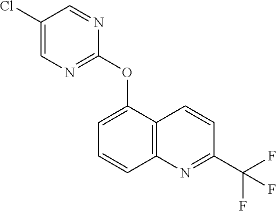 [SMILES: FC(F)(F)c1ccc2c(Oc3ncc(Cl)cn3)cccc2n1] | 8.51 (s, 2 H), 8.49 (d, 1 H), 8.20 (d, 1 H), 7.88 (t, 1 H), 7.74 (d, 1 H), 7.52 (d, 1 H) |
| 1.021 | 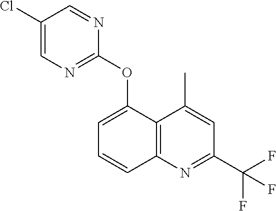 [SMILES: Cc1cc(C(F)(F)F)nc2cccc(Oc3ncc(Cl)cn3)c12] | 8.51 (s, 2 H), 8.20 (dd, 1 H), 7.81 (t, 1 H), 7.51 (s, 1 H), 7.38 (d, 1 H), 2.79 (s, 3 H) |
| 1.022 | 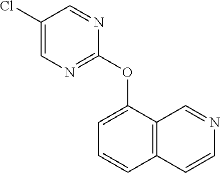 [SMILES: Clc1cnc(Oc2cccc3ccncc23)nc1] | 9.35 (s, 1 H), 8.58 (d, 1 H), 8.49 (s, 2 H), 7.69- 7.77 (m, 3 H), 7.39 (dd, 1 H) |
| 1.023 | 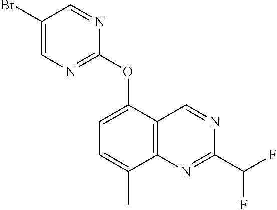 [SMILES: Cc1ccc(Oc2ncc(Br)cn2)c2cnc(C(F)F)nc12] | 9.60 (s, 1 H), 8.59 (s, 2 H), 7.88 (d, 1 H), 7.45 (d, 1 H), 6.84 (t, 1 H), 2.82 (s, 3 H) |
| 1.024 | 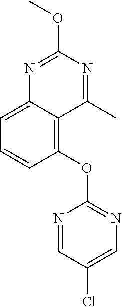 [SMILES: COc1nc(C)c2c(Oc3ncc(Cl)cn3)cccc2n1] | 8.52 (s, 2 H), 7.77-7.79 (m, 2 H), 7.10-7.15 (m, 1 H), 4.12 (s, 3 H), 2.85 (s, 3 H) |
| 1.025 | 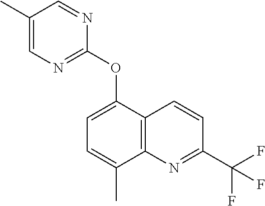 [SMILES: Cc1cnc(Oc2ccc(C)c3nc(C(F)(F)F)ccc23)nc1] | 8.46 (d, 1 H), 8.37 (s, 2 H), 7.70 (d, 2 H), 7.39 (d, 1 H), 2.85 (s, 3 H), 2.28 (s, 3 H) |
| 1.026 | 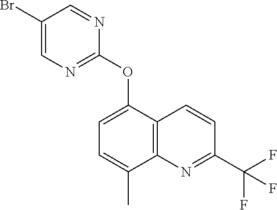 [SMILES: Cc1ccc(Oc2ncc(Br)cn2)c2ccc(C(F)(F)F)nc12] | (400 MHz, DMSO-d6) 8.81 (s, 2 H), 8.54 (d, 1 H), 7.95 (d, 1 H), 7.85 (d, 1 H), 7.61 (d, 1 H), 2.76 (s, 3 H) |
| 1.027 | 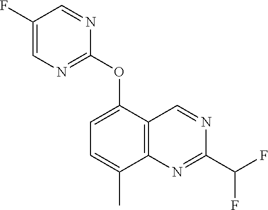 [SMILES: Cc1ccc(Oc2ncc(F)cn2)c2cnc(C(F)F)nc12] | 9.62 (s, 1 H) 8.44 (s, 2 H) 7.86 (d, 1 H) 7.45 (d, 1 H) 6.84 (s, 1 H) 2.82 (s, 3 H) |
| 1.028 | 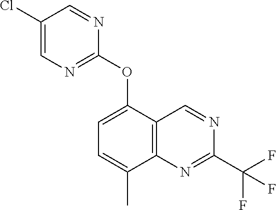 [SMILES: Cc1ccc(Oc2ncc(Cl)cn2)c2cnc(C(F)(F)F)nc12] | 9.60 (s, 1 H) 8.51 (s, 2 H) 7.87 (d, 1 H) 7.45 (d, 1 H) 6.84 (t, 1 H) 2.82 (s, 3 H) |
| 1.029 | 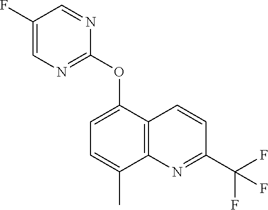 [SMILES: Cc1ccc(Oc2ncc(F)cn2)c2ccc(C(F)(F)F)nc12] | (400 MHz, DMSO-d6) 8.74 (s, 2 H), 8.54 (d, 1 H), 7.96 (d, 1 H), 7.85 (d, 1 H), 7.59 (d, 1 H), 2.76 (s, 3 H) |
| 1.030 | 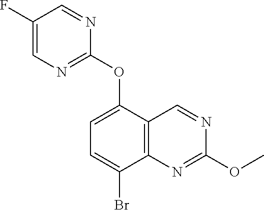 [SMILES: COc1ncc2c(Oc3ncc(F)cn3)ccc(Br)c2n1] | 9.37 (s, 1 H), 8.44 (s, 2 H), 8.13 (d, 1 H), 7.14 (d, 1 H), 4.21 (s, 3 H) |
| 1.031 | 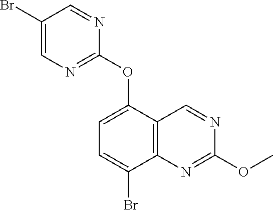 [SMILES: COc1ncc2c(Oc3ncc(Br)cn3)ccc(Br)c2n1] | 9.35 (s, 1 H), 8.60 (s, 2 H), 8.14 (d, 1 H), 7.16 (d, 1 H), 4.22 (s, 3 H) |
| 1.032 | 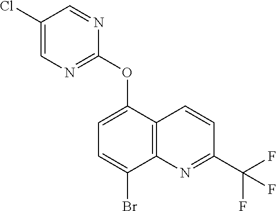 [SMILES: FC(F)(F)c1ccc2c(Oc3ncc(Cl)cn3)ccc(Br)c2n1] | (400 MHz, DMSO-d6) 8.80 (s, 2 H), 8.68 (d, 1 H), 8.41 (d, 1 H), 8.07 (d, 1 H), 7.69 (d, 1 H) |
| 1.033 | 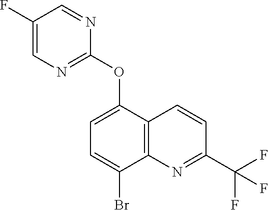 [SMILES: Fc1cnc(Oc2ccc(Br)c3nc(C(F)(F)F)ccc23)nc1] | (400 MHz, DMSO-d6) 8.77 (s, 2 H), 8.67 (d, 1 H), 8.39 (d, 1 H), 8.07 (d, 1 H), 7.66 (d, 1 H) |
| 1.034 | 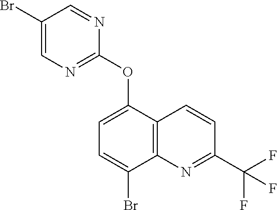 [SMILES: FC(F)(F)c1ccc2c(Oc3ncc(Br)cn3)ccc(Br)c2n1] | (400 MHz, DMSO-d6) 8.84 (s, 2 H), 8.67 (d, 1 H), 8.40 (d, 1 H), 8.06 (d, 1 H), 7.69 (d, 1 H) |
| 1.035 | 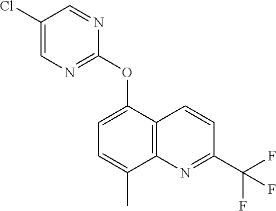 [SMILES: Cc1ccc(Oc2ncc(Cl)cn2)c2ccc(C(F)(F)F)nc12] | (400 MHz, DMSO-d6) 8.77 (s, 2 H), 8.55 (d, 1 H), 7.96 (d, 1 H), 7.86 (d, 1 H), 7.62 (d, 1 H), 2.77 (s, 3 H) |
| 1.036 | 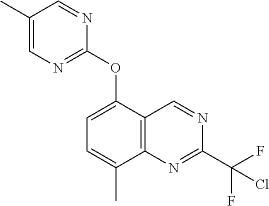 [SMILES: Cc1cnc(Oc2ccc(C)c3nc(C(F)(F)Cl)ncc23)nc1] | 9.62 (s, 1 H), 8.39 (s, 2 H), 7.89 (d, 1 H), 7.48 (d, 1 H), 2.83 (s, 3 H), 2.31 (s, 3H) |
| 1.037 | 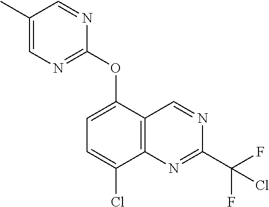 [SMILES: Cc1cnc(Oc2ccc(Cl)c3nc(C(F)(F)Cl)ncc23)nc1] | 9.72 (s, 1 H), 8.41 (s, 2 H), 8.14 (d, 1 H), 7.55 (d, 1 H), 2.33 (s, 3 H) |
| 1.038 | 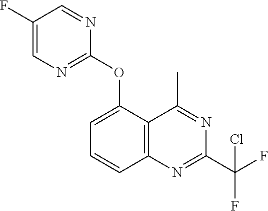 [SMILES: Cc1nc(C(F)(F)Cl)nc2cccc(Oc3ncc(F)cn3)c12] | 8.46 (s, 2H) 8.13 (dd, 1H) 8.00 (t, 1 H) 7.48 (dd, 1 H) 3.03 (s, 3 H) |
| 1.039 | 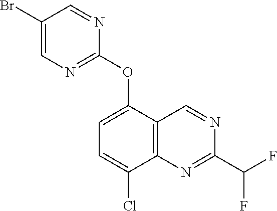 [SMILES: FC(F)c1ncc2c(Oc3ncc(Br)cn3)ccc(Cl)c2n1] | 9.69 (s, 1 H), 8.62 (s, 2 H), 8.13 (d, 1 H), 7.54 (d, 1 H), 6.90 (t, 1 H) |
| 1.040 | 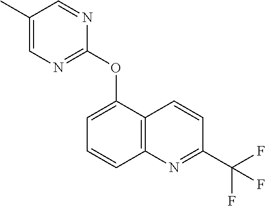 [SMILES: Cc1cnc(Oc2cccc3nc(C(F)(F)F)ccc23)nc1] | 8.52 (d, 1 H), 8.38 (s, 2 H), 8.16 (d, 1 H), 7.86 (dd, 1 H), 7.71 (d, 1 H), 7.50 (d, 1 H), 2.29 (s, 3 H) |
| 1.041 | 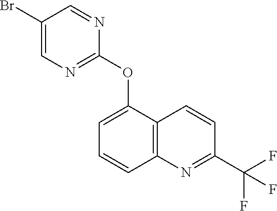 [SMILES: FC(F)(F)c1ccc2c(Oc3ncc(Br)cn3)cccc2n1] | 8.58 (s, 2 H), 8.48 (d, 1 H), 8.20 (d, 1 H), 7.87 (dd, 1 H), 7.74 (d, 1 H), 7.51 (d, 1 H) |
| 1.042 | 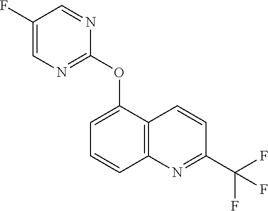 [SMILES: Fc1cnc(Oc2cccc3nc(C(F)(F)F)ccc23)nc1] | 8.43 (d, 1H), 8.36 (s, 2 H), 8.12 (d, 1 H), 7.80 (dd, 1 H), 7.66 (d, 1 H), 7.44 (dd, 1 H) |
| 1.043 | 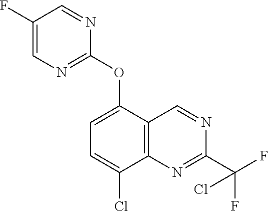 [SMILES: Fc1cnc(Oc2ccc(Cl)c3nc(C(F)(F)Cl)ncc23)nc1] | 9.72 (s, 1 H), 8.47 (s, 2 H), 8.16 (d, 1 H), 7.56 (d, 1 H) |
| 1.044 | 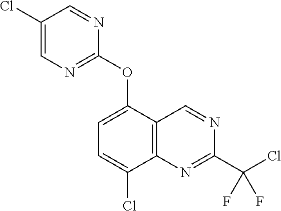 [SMILES: FC(F)(Cl)c1ncc2c(Oc3ncc(Cl)cn3)ccc(Cl)c2n1] | 9.70 (s, 1 H), 8.54 (s, 2 H), 8.16 (d, 1 H), 7.57 (d, 1 H) |
| 1.045 | 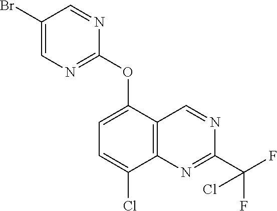 [SMILES: FC(F)(Cl)c1ncc2c(Oc3ncc(Br)cn3)ccc(Cl)c2n1] | 9.70 (s, 1 H), 8.62 (s, 2 H), 8.16 (d, 1 H), 7.57 (d, 1 H) |
| 1.046 | 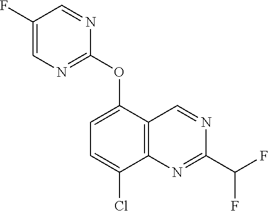 [SMILES: Fc1cnc(Oc2ccc(Cl)c3nc(C(F)F)ncc23)nc1] | 9.71 (s, 1 H), 8.46 (s, 2 H), 8.13 (d, 1 H), 7.54 (d, 1 H), 6.90-6.76 (t, 1 H) |
| 1.047 | 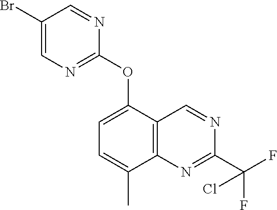 [SMILES: Cc1ccc(Oc2ncc(Br)cn2)c2cnc(C(F)(F)Cl)nc12] | 9.60 (s, 1 H), 8.60 (s, 2 H), 7.90 (d, 1 H), 7.48 (d, 1 H), 2.84 (s, 3 H) |
| 1.048 | 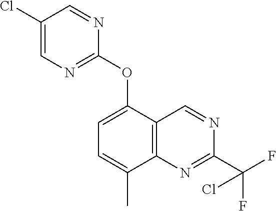 [SMILES: Cc1ccc(Oc2ncc(Cl)cn2)c2cnc(C(F)(F)Cl)nc12] | 9.61 (s, 1 H), 8.52 (s, 2 H), 7.90 (d, 1 H), 7.49 (d, 1 H), 2.84 (s, 3H) |
| 1.049 | 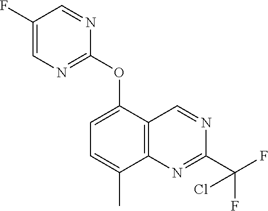 [SMILES: Cc1ccc(Oc2ncc(F)cn2)c2cnc(C(F)(F)Cl)nc12] | 9.63 (s, 1 H), 8.44 (s, 2 H), 7.90 (d, 1 H), 7.48 (d, 1 H), 2.84 (s, 3H) |
| 1.050 | 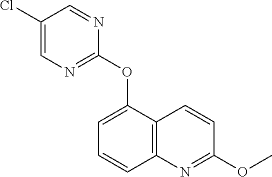 [SMILES: COc1ccc2c(Oc3ncc(Cl)cn3)cccc2n1] | (400 MHz, DMSO-d6) 8.76 (s, 2 H), 8.08 (d, 1 H), 7.70-7.74 (m, 2 H), 7.31 (dd, 1 H), 7.01 (d, 1 H), 4.06 (s, 3H) |
| 1.051 | 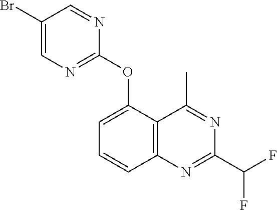 [SMILES: Cc1nc(C(F)F)nc2cccc(Oc3ncc(Br)cn3)c12] | 8.61 (s, 2 H), 8.10 (dd, 1 H), 7.98 (t, 1 H), 7.45 (dd, 1 H), 6.76 (t, 1 H), 3.00 (s, 3 H) |
| 1.052 | 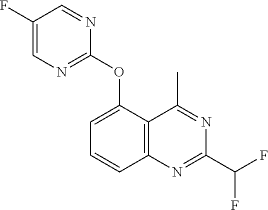 [SMILES: Cc1nc(C(F)F)nc2cccc(Oc3ncc(F)cn3)c12] | 8.46 (s, 2 H), 8.09 (dd, 1 H), 7.97 (t, 1 H), 7.45 (dd, 1 H), 6.76 (t, 1 H), 3.01 (s, 3 H) |
| 1.053 | 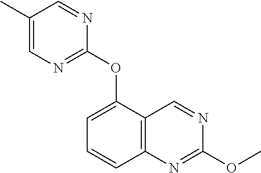 | 9.38 (s, 1 H), 8.40 (s, 2 H), 7.77-7.89 (m, 1 H), 7.73 (d, 1 H), 7.23 (dd, 1 H), 4.15 (s, 3 H), 2.30 (s, 3 H) |
| 1.054 | 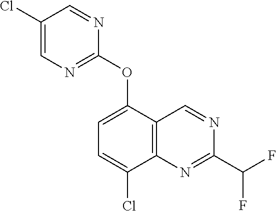 [SMILES: FC(F)c1ncc2c(Oc3ncc(Cl)cn3)ccc(Cl)c2n1] | 9.71 (s, 1 H), 8.55 (s, 2 H), 8.15 (d, 1 H), 7.55 (d, 1 H), 6.91 (t, 1 H) |
| 1.055 | 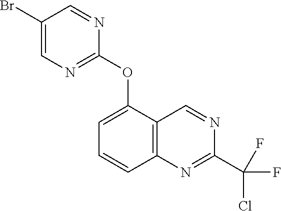 [SMILES: FC(F)(Cl)c1ncc2c(Oc3ncc(Br)cn3)cccc2n1] | 9.69 (s, 1 H), 8.63 (s, 2 H), 8.08-8.17 (m, 2 H), 7.62 (dd, 1 H) |
| 1.056 | 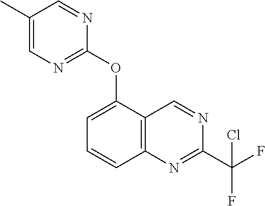 [SMILES: Cc1cnc(Oc2cccc3nc(C(F)(F)Cl)ncc23)nc1] | 9.70 (s, 1 H), 8.41 (s, 2 H), 8.05-8.13 (m, 2 H), 7.59 (dd, 1 H), 2.32 (s, 3 H) |
| 1.057 | 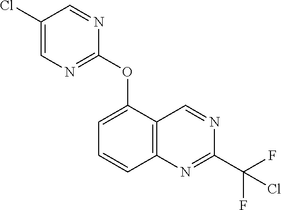 [SMILES: FC(F)(Cl)c1ncc2c(Oc3ncc(Cl)cn3)cccc2n1] | 9.67 (s, 1 H) 8.53 (s, 2 H) 8.06-8.15 (m, 2 H) 7.60 (dd, 1 H) |
| 1.058 | 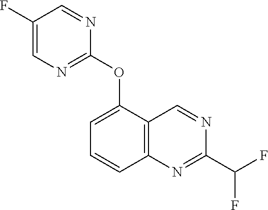 [SMILES: Fc1cnc(Oc2cccc3nc(C(F)F)ncc23)nc1] | 9.68 (s, 1 H), 8.46 (s, 2 H), 8.03-8.12 (m, 2 H), 7.57 (dd, 1 H), 6.84 (t, 1 H) |
| 1.059 | 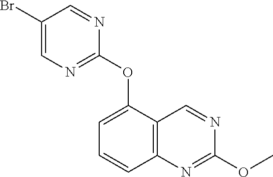 [SMILES: COc1ncc2c(Oc3ncc(Br)cn3)cccc2n1] | 9.35 (s, 1 H), 8.60 (s, 2 H), 7.82-7.92 (m, 1 H), 7.73-7.81 (m, 1 H), 7.24 (dd, 1 H), 4.15 (s, 3 H) |
| 1.060 | 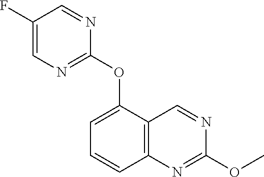 [SMILES: COc1ncc2c(Oc3ncc(F)cn3)cccc2n1] | δ = 9.36 (s, 1 H), 8.44 (s, 2 H), 7.81-7.88 (m, 1 H), 7.69-7.78 (m, 1 H), 7.22 (dd, 1 H), 4.14 (s, 3 H) |
| 1.061 | 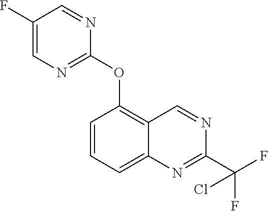 [SMILES: Fc1cnc(Oc2cccc3nc(C(F)(F)Cl)ncc23)nc1] | 9.69 (s, 1 H), 8.45 (s, 2 H), 8.05-8.14 (m, 2 H), 7.59 (dd, 1 H). |
| 1.062 | 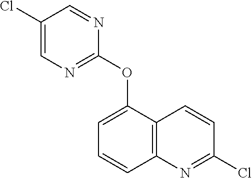 [SMILES: Clc1cnc(Oc2cccc3nc(Cl)ccc23)nc1] | (400 MHz, DMSO-d6) 8.78 (s, 2 H) 8.29-8.33 (m, 1 H) 7.88-7.96 (m, 2H) 7.56-7.61 (m, 2H) |
| 1.063 | 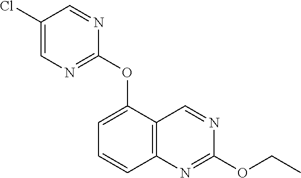 [SMILES: CCOc1ncc2c(Oc3ncc(Cl)cn3)cccc2n1] | 9.34 (s, 1 H), 8.52 (s, 2 H), 7.84 (dd, 1 H), 7.74 (d, 1 H), 7.22 (d, 1 H), 4.58 (q, 2 H), 1.45-1.55 (m, 3 H) |
| 1.064 | 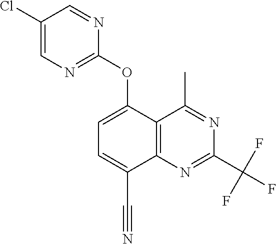 [SMILES: Cc1nc(C(F)(F)F)nc2c(C#N)ccc(Oc3ncc(Cl)cn3)c12] | 8.58 (s, 2 H) 8.39 (d, 1 H) 7.59 (d, 1 H) 3.11 (s, 3 H) |
| 1.065 | 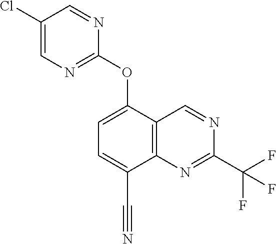 [SMILES: N#Cc1ccc(Oc2ncc(Cl)cn2)c2cnc(C(F)(F)F)nc12] | (400 MHz, METHANOL-d4) 9.98 (s, 1 H), 8.73 (s, 2 H), 8.67 (d, 1 H), 7.95 (d, 1 H) |
| 1.066 | 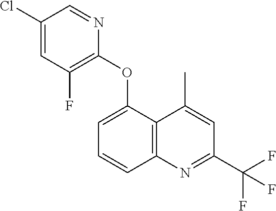 [SMILES: Cc1cc(C(F)(F)F)nc2cccc(Oc3ncc(Cl)cc3F)c12] | 8.15 (dd, 1 H), 7.87 (d, 1 H), 7.78 (t, 1 H), 7.61 (dd, 1H), 7.51 (d, 1 H), 7.31 (dd, 1 H), 2.84 (s, 3 H) |
| 1.067 | 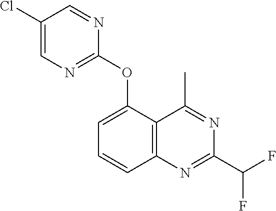 [SMILES: Cc1nc(C(F)F)nc2cccc(Oc3ncc(Cl)cn3)c12] | 8.54 (s, 2 H), 8.11 (d, 1 H), 7.99 (t, 1 H), 7.47 (d, 1 H), 6.91-6.64 (t, 1 H), 3.02 (s, 3 H) |
| 1.068 | 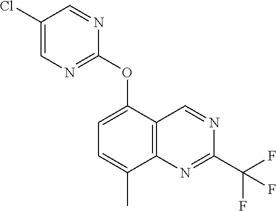 [SMILES: Cc1ccc(Oc2ncc(Cl)cn2)c2cnc(C(F)(F)F)nc12] | 9.60 (s, 1 H), 8.51 (s, 2 H), 7.90 (d, 1 H), 7.49 (d, 1 H), 2.83 (s, 3 H) |
| 1.069 | 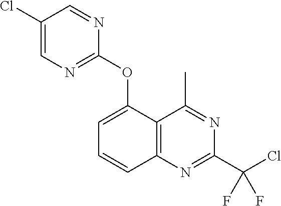 [SMILES: Cc1nc(C(F)(F)Cl)nc2cccc(Oc3ncc(Cl)cn3)c12] | 8.53 (s, 2 H), 8.13 (dd, 1 H), 8.00 (dd, 1 H), 7.48 (dd, 1 H), 3.02 (s, 3 H) |
| 1.070 | 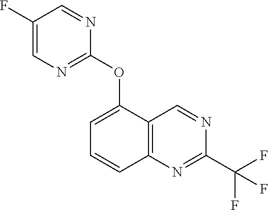 [SMILES: Fc1cnc(Oc2cccc3nc(C(F)(F)F)ncc23)nc1] | 9.70 (s, 1 H), 8.45 (s, 2 H), 8.05-8.16 (m, 2 H), 7.61 (dd, 1 H) |
| 1.071 | 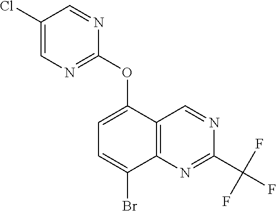 [SMILES: FC(F)(F)c1ncc2c(Oc3ncc(Cl)cn3)ccc(Br)c2n1] | 9.68 (s, 1 H), 8.53 (s, 2 H), 8.37 (d, 1 H), 7.52 (d, 1 H) |
| 1.072 | 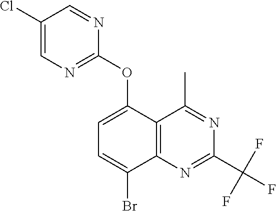 [SMILES: Cc1nc(C(F)(F)F)nc2c(Br)ccc(Oc3ncc(Cl)cn3)c12] | 8.52 (s, 2 H), 8.31 (d, 1 H), 7.38 (d, 1 H), 3.03 (s, 3 H) |
| 1.073 | 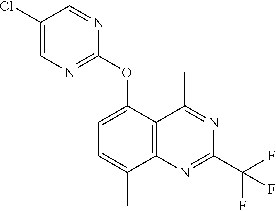 [SMILES: Cc1ccc(Oc2ncc(Cl)cn2)c2c(C)nc(C(F)(F)F)nc12] | 8.51 (s, 2 H), 7.84 (dd, 1 H), 7.37 (d, 1 H), 2.98 (s, 3 H), 2.81 (s, 3 H) |

### Biological Examples

Seeds of a variety of test species are sown in standard soil in pots Amaranthus retoflexus (AMARE), Amaranthus tuberculatus (AMATA), Echinochloa crus-galli (ECHCG), Ipomoea hederacea (IPOHE) and Setaria faberi (SETFA). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| Compound | AMARE | AMAPA | ECHCG | IPOHE | SETFA |
|---|---|---|---|---|---|
| 1.001 | 5 | 5 | 3 | 2 | 4 |
| 1.002 | 5 | 5 | 5 | 4 | 4 |
| 1.003 | 1 | 1 | 1 | 1 | 1 |
| 1.004 | 3 | 4 | 3 | 4 | 4 |
| 1.005 | 4 | 5 | 4 | 4 | 4 |
| 1.006 | 2 | 1 | 1 | 2 | 1 |
| 1.007 | 2 | — | 3 | 2 | 4 |
| 1.008 | 3 | 3 | 1 | 3 | 1 |
| 1.009 | 1 | 1 | 1 | 1 | 1 |
| 1.012 | 1 | 1 | 1 | 1 | — |
| 1.016 | 4 | 5 | 1 | 2 | 1 |
| 1.019 | 4 | 3 | 2 | 4 | 3 |
| 1.020 | 5 | 5 | 4 | 4 | 5 |
| 1.021 | 3 | 2 | 1 | 2 | 1 |
| 1.022 | 1 | 2 | 1 | 1 | 1 |
| 1.023 | 2 | 3 | 1 | 1 | — |
| 1.024 | 1 | 2 | 1 | 1 | — |
| 1.025 | 2 | 3 | 1 | 2 | — |
| 1.026 | 2 | 2 | 1 | 1 | — |
| 1.027 | 2 | 2 | 1 | 2 | — |
| 1.028 | 2 | 3 | 1 | 2 | — |
| 1.029 | 3 | 4 | 1 | 2 | — |
| 1.030 | 2 | 3 | 1 | 1 | — |
| 1.031 | 1 | 1 | 1 | 1 | — |
| 1.032 | 2 | 2 | 1 | 1 | — |
| 1.033 | 2 | 1 | 1 | 1 | — |
| 1.034 | 1 | 1 | 1 | 1 | — |
| 1.035 | 3 | 3 | 1 | 2 | — |
| 1.036 | 2 | 3 | 1 | 1 | — |
| 1.037 | 2 | 2 | 1 | 1 | — |
| 1.038 | 4 | 5 | 3 | 3 | — |
| 1.039 | 2 | 2 | 1 | 1 | — |
| 1.040 | 4 | 4 | 4 | 5 | 4 |
| 1.041 | 5 | 5 | 2 | 3 | 4 |
| 1.042 | 5 | 5 | 4 | 4 | — |
| 1.043 | 3 | 3 | 1 | 2 | — |
| 1.044 | 3 | 3 | 1 | 2 | 3 |
| 1.046 | 2 | 1 | 1 | 2 | — |
| 1.047 | 2 | 2 | 1 | 2 | 1 |
| 1.048 | 2 | 2 | 1 | 1 | — |
| 1.049 | 2 | 3 | 1 | 2 | — |
| 1.051 | 1 | 1 | 1 | 1 | — |
| 1.052 | 1 | 1 | 1 | 2 | — |
| 1.054 | 2 | 2 | 1 | 1 | — |
| 1.055 | 2 | 3 | 2 | 2 | — |
| 1.056 | 2 | 4 | 3 | 4 | — |
| 1.057 | 4 | 4 | 4 | 4 | — |
| 1.058 | 2 | 2 | 1 | 4 | — |
| 1.061 | 4 | 4 | 4 | 4 | — |
| 1.063 | 5 | 5 | 5 | 5 | 4 |
| 1.064 | 1 | 1 | 1 | 1 | 1 |
| 1.065 | 1 | 1 | 1 | 2 | — |
| 1.066 | 1 | 1 | 1 | 1 | — |
| 1.067 | 4 | 4 | 1 | 3 | — |
| 1.068 | 3 | 2 | 1 | 2 | 1 |
| 1.069 | 3 | 3 | 2 | 2 | 2 |
| 1.070 | 4 | 4 | 4 | 4 | 4 |
| 1.071 | 3 | 2 | 1 | 2 | 1 |
| 1.072 | 1 | 1 | 1 | 1 | 1 |
| 1.073 | 1 | 1 | 1 | 1 | 1 |

TABLE B2
Pre-emergence Test

| Compound | AMARE | AMAPA | ECHCG | IPOHE | SETFA |
|---|---|---|---|---|---|
| 1.001 | 5 | 5 | 3 | 5 | 3 |
| 1.002 | 5 | 5 | 5 | 5 | 5 |
| 1.003 | 1 | 1 | 1 | 1 | 1 |
| 1.004 | 5 | 4 | 5 | 4 | 4 |
| 1.005 | 5 | 5 | 3 | 3 | 4 |
| 1.006 | 1 | 1 | 1 | 1 | 1 |
| 1.007 | 4 | 4 | 4 | 2 | 5 |
| 1.008 | 4 | 4 | 1 | 3 | 4 |
| 1.009 | 1 | 2 | 1 | 1 | 1 |
| 1.012 | 1 | 1 | 1 | 1 | — |
| 1.016 | 5 | 5 | 3 | 4 | 3 |
| 1.019 | 5 | 5 | 5 | 4 | 5 |
| 1.020 | 5 | 5 | 5 | 4 | 5 |
| 1.021 | 1 | 1 | 1 | 1 | 1 |
| 1.022 | 1 | 1 | 1 | 1 | 1 |
| 1.023 | 2 | 1 | 1 | 1 | — |
| 1.024 | 3 | 3 | 1 | 1 | — |
| 1.025 | 1 | 1 | 1 | 1 | — |
| 1.026 | 1 | 1 | 1 | 1 | — |
| 1.027 | 2 | 2 | 1 | 1 | — |
| 1.028 | 5 | 5 | 2 | 1 | — |
| 1.029 | 2 | 3 | 1 | 1 | — |
| 1.030 | 1 | 1 | 1 | 1 | — |
| 1.031 | 1 | 1 | 1 | 1 | — |
| 1.032 | 1 | 1 | 1 | 1 | — |
| 1.033 | 3 | 3 | 1 | 1 | — |
| 1.034 | 1 | 1 | 1 | 1 | — |
| 1.035 | 2 | 2 | 1 | 1 | — |
| 1.036 | 4 | 3 | 1 | 1 | — |
| 1.037 | 5 | 4 | 1 | 1 | — |
| 1.038 | 5 | 5 | 4 | 5 | — |
| 1.039 | 1 | 1 | 1 | 1 | — |
| 1.040 | 5 | 5 | 5 | 5 | 5 |
| 1.041 | 5 | 5 | 3 | 2 | 4 |
| 1.042 | 5 | 5 | 5 | 5 | — |
| 1.043 | 4 | 4 | 1 | 1 | — |
| 1.044 | 3 | 5 | 2 | 1 | 5 |
| 1.046 | 5 | 4 | 1 | 1 | — |
| 1.047 | 2 | 2 | 1 | 1 | 1 |
| 1.048 | — | 1 | 1 | 1 | — |
| 1.049 | 1 | 1 | 1 | 1 | — |
| 1.051 | 1 | 1 | 1 | 1 | — |
| 1.052 | 2 | 1 | 2 | 1 | — |
| 1.054 | 5 | 5 | 2 | 1 | — |
| 1.055 | 5 | 5 | 4 | 2 | — |
| 1.056 | 5 | 5 | 5 | 5 | — |
| 1.057 | 5 | 5 | 5 | 5 | — |
| 1.058 | 5 | 5 | 1 | 3 | — |
| 1.061 | 5 | 5 | 5 | 5 | — |
| 1.063 | 5 | 5 | 5 | 5 | 4 |
| 1.064 | 1 | 1 | 1 | 1 | 1 |
| 1.065 | 1 | 1 | 1 | 1 | — |
| 1.066 | 1 | 1 | 1 | 1 | — |
| 1.067 | | 3 | 1 | 2 | — |
| 1.068 | 5 | 5 | 3 | 2 | 3 |
| 1.069 | 5 | 5 | 2 | 1 | 5 |
| 1.070 | 5 | 5 | 5 | 5 | 5 |
| 1.071 | 3 | 5 | 3 | 1 | 4 |
| 1.072 | 1 | 2 | 1 | 1 | 1 |
| 1.073 | 1 | 1 | 1 | 1 | 1 |

1. A compound of Formula (I): 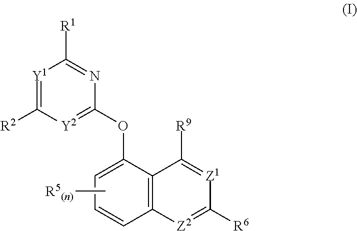
or an agronomically acceptable salt thereof,
wherein
Y¹ is N or CR³;
Y² is N or CR⁴;
with the proviso that Y¹ and Y² are not both N;
R¹ is selected from the group consisting of hydrogen, halogen, C₁-C₃alkyl and C₁-C₃haloalkyl;
R² is selected from the group consisting of hydrogen, halogen, hydroxyl, C₁-C₃alkyl, C₁-C₃alkoxy-, C₁-C₃haloalkoxy- and C₁-C₃haloalkyl;
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
each R⁵ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁶ is independently selected from the group consisting of hydrogen, halogen, —CN, hydroxy, C₁-C₆alkyl, C₁-C₆haloalkyl-, C₁-C₆alkoxy-, C₁-C₆haloalkoxy- and C₃-C₆cycloalkyl-;
Z¹ is N or CR⁷;
Z² is N or CR⁸;
R⁷ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁸ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁹ is hydrogen or methyl; and
n=0, 1 or 2.

2. A compound according to claim 1, wherein R¹ and R² are both hydrogen.

3. A compound according to claim 1, wherein Y¹ is CR³ and Y² is N.

4. A compound according to claim 3, wherein R³ is chloro.

5. A compound according to claim 1, wherein n=0.

6. A compound according to claim 1, wherein R⁶ is selected from the group consisting of hydrogen, C₁-C₆alkyl, C₁-C₆alkoxy- and C₁-C₆haloalkyl-.

7. A compound according to claim 6, wherein R⁶ is selected from the group consisting of ethoxy-, —CClF₂ and CF₃.

8. A compound according to claim 1, wherein Z¹ is CH and Z² is CH.

9. A compound according to claim 1, wherein Z¹ is N and Z² is N.

10. A compound according to claim 1, wherein R⁹ is hydrogen.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as a herbicide.
